(19) United States
(12) Patent Application Publication
(10) Pub. No.: US 2026/0356984 A1
(43) Pub. Date: Nov. 12, 2026

(54) DNA-AFFIBODY-DRUG NANOSTRUCTURES FOR INHIBITING TRIPLE NEGATIVE BREAST CANCER GROWTH AND METASTASIS

(71) Applicants: ARIZONA BOARD OF REGENTS ON BEHALF OF ARIZONA STATE UNIVERSITY, Scottsdale, AZ (US); CEDARS-SINAI MEDICAL CENTER, Los Angeles, CA (US)

(72) Inventors: Shengxi CHEN, Chandler, AZ (US); Xiaojiang CUI, Arcadia, CA (US); Yi ZHANG, Los Angeles, CA (US); Sidney HECHT, Phoenix, AZ (US)

(21) Appl. No.: 19/115,431

(22) PCT Filed: Sep. 26, 2023
(86) PCT No.: PCT/US2023/075103

§ 371 Date: Mar. 26, 2025

Related U.S. Application Data
(60) Provisional application No. 63/410,187, filed on Sep. 26, 2022.

(51) Int. Cl. C07K 19/00; A61K 31/337; A61K 31/453; A61K 31/454; A61K 31/506; A61K 31/519; A61K 31/52; A61K 31/704; A61K 39/395; A61P 35/00; C07K 16/28; C07K 16/32

(52) U.S. Cl. CPC C07K 19/00; A61K 31/337; A61K 31/453; A61K 31/454; A61K 31/506; A61K 31/519; A61K 31/52; A61K 31/704; A61K 39/39558; A61P 35/00; C07K 16/2863; C07K 16/32; C07K 2318/00

(57) ABSTRACT

Provided herein are nanostructures comprising peptides specific for the extracellular domains of HER1 and/or HER3. Also provided herein are methods for use of the nanostructures to aggregate cells and deliver therapeutic agents to the cells. The nanostructures and methos are useful for treating and/or preventing cancers or other conditions associated with HER1 and/or HER3 overexpression.

### CROSS-REFERENCE TO RELATED APPLICATIONS

This application claims priority to U.S. Provisional Application No. 63/410,187 filed on Sep. 26, 2022, the content of which is incorporated by reference in its entirety.

### STATEMENT REGARDING FEDERALLY SPONSORED RESEARCH

This invention was made with government support under R21 CA280458 awarded by the National Institutes of Health. The government has certain rights in the invention.

### SEQUENCE LISTING

A Sequence Listing accompanies this application and is submitted as an xml file of the sequence listing named “112624.01426.xml” which is 23,277 bytes in size and was created on Sep. 26, 2023. The sequence listing is electronically submitted via Patent Center with the application and is incorporated herein by reference in its entirety.

### BACKGROUND

Triple-negative breast cancer (TNBC), a subtype of breast cancer that lacks estrogen receptor, progresterone, and HER2 expression, is associated with poor prognosis and a high lung and brain metastasis rate. TNBC is highly invasive, and approximately 46% of TNBC patients will have distant metastasis, which is the major cause of death from this disease. The standard treatment options for TNBC include surgery, radiotherapy, and chemotherapy, individually or in combination. Chemotherapeutic drugs have limited efficacy in inhibiting cancer growth, and generally have high toxicity in patients. Therefore, there is an urgent need to develop new agents to specifically target TNBC to inhibit its growth and metastasis.

The HER1 and/or HER3 receptors are overexpressed in most TNBCs. Inappropriate activation of these receptors is associated with the development, metastasis, and therapeutic resistance of many cancers. Currently, several antibodies and antibody-conjugate agents targeting the HER 1 and HER3 receptors (called targeted therapy) are being developed for TNBC treatment. Although clinical trials have not yielded positive results, the HER1-targeting approach is still of particular interest since HER1 overexpression potentiates key functions of cancer cells. Several studies have confirmed that HER1 is an important target in TNBC, in which at least 50% of cases overexpress HER1. Anti-HER1 monoclonal antibodies (e.g. cetuximab and panitumumab) have been approved by FDA to treat metastatic colorectal cancer, metastatic non-small cell lung cancer, and head and neck cancer. Neither antibody confers significant benefit to TNBC patients when employed as a single agent. Nevertheless, HER1-targeting antibodies can enhance the chemosensitivity of TNBC cells in patients. HER3 has also recently gained more attention as a therapeutic target for developing a new therapy for TNBC. Patritumab deruxtecan (U3-1402) is an antibody drug conjugate, which demonstrated early and clinically meaningful activity for breast cancer patients, and is currently being evaluated in a phase II trial. However, to date, no first-line targeted therapy for TNBC treatment has been successfully developed, and few existing treatment modalities can effectively block the metastasis of TNBC.

Small molecule drugs have been attractive agents for cancer treatment for many years because of their small size, oral availability, ability to cross membranes, and low cost. However, small molecules are known to have limitations, such as low specificity and unacceptable toxicity. Accordingly, there remains a need for improved therapeutic compositions and therapeutic strategies for treating TNBC. In particular, there remains a need for improved therapeutic compositions and methods for treating cancers, including metastatic cancers, associated with overexpression of HER 1 and/or HER3.

### SUMMARY

In an aspect, provided herein is a nanostructure comprising a tetrahedron-shaped polynucleotide; and four peptides linked to the polynucleotide, wherein each of the peptides are selected from a HER1 binding peptide and a HER3 binding peptide. In embodiments, the nanostructure comprises four HER1 binding peptides. In embodiments, the nanostructure comprises four HER3 binding peptides. In embodiments, the tetrahedron-shaped polynucleotide comprises four polynucleotide strands, wherein the sequences of the four polynucleotide strands individually comprise SEQ ID NOs: 12, 13, 14, and 15. In embodiments, the HER1 binding peptide comprises SEQ ID NO: 10, or a sequence having at least 95% identity thereto. In embodiments, the HER3 binding peptide comprises SEQ ID NO: 11, or a sequence having at least 95% identity thereto. In embodiments, each of the peptides are affibodies. In embodiments, the polynucleotide is DNA or RNA.

In embodiments, the nanostructure further comprises a plurality of molecules of at least one drug. In embodiments, the drug is a CDK7 inhibitor. In embodiments, the drug is THZ1. In embodiments, the nanostructure comprises between 5 and 1,000 molecules of THZ1. In embodiments, the nanostructure comprises between 10 and 50 molecules of THZ1. In embodiments, the at least one drug is selected from the group consisting of doxorubicin, epirubicin, docetaxel, ribociclib, THZ2, Alvocidib, Seliciclib, and SNS-032.

In another aspect, provided herein is a pharmaceutical composition comprising any of the nanostructures disclosed herein and a pharmaceutically acceptable carrier.

In another aspect, provided herein is a method of treating a cancer associated with overexpression of at least one of HER1 and HER3 in a subject in need thereof, the method comprising administering to the subject a therapeutically effective amount of the pharmaceutical composition disclosed herein. In embodiments, treating the cancer comprises reducing metastasis of the cancer. In embodiments, treating the cancer comprises reducing growth of the cancer. In embodiments, the cancer is triple negative breast cancer.

In another aspect, provided herein is a method of crosslinking cancer cells overexpressing at least one of HER1 and HER3, the method comprising treating the cells with any of the nanostructures disclosed herein.

### BRIEF DESCRIPTION OF THE DRAWINGS

FIG. 1. Structures of nanostructures I and II. I illustrates a drug-loaded DNA tetrahedron with four molecules of an affibody at the apexes. II illustrates a drug-loaded DNA tetrahedron with four molecules of an affibody at the edges.

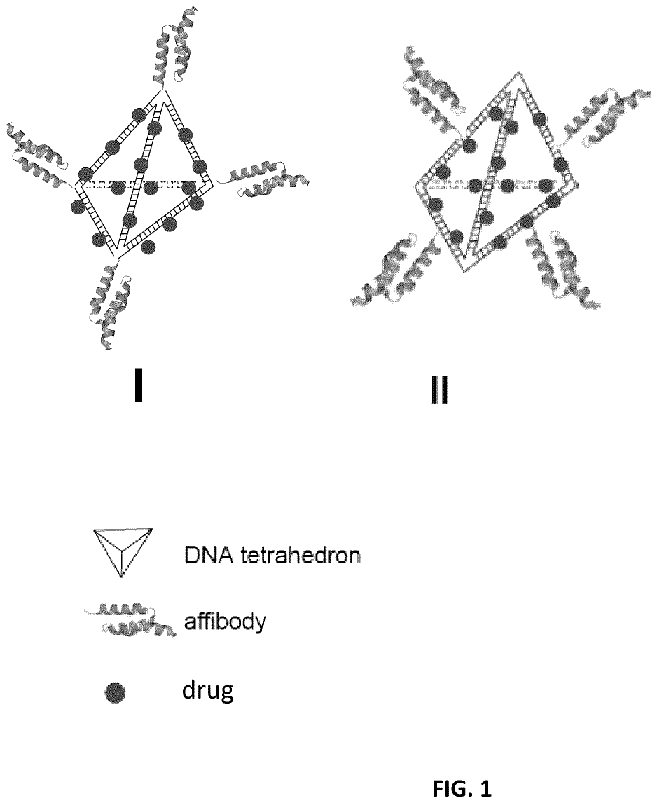

FIG. 2. Structure of THZ1.

FIGS. 3A-3B. DNA-THZ1 binding assay. (A) Analysis of binding ratio of THZ1 to DNA tetrahedron using UV absorption of THZ1 (220 nm). DNA tetrahedron (0.2 μM) was added different amounts of THZ1 (2-12 μM). The unbound THZ1 was removed through a Sephadex G-25 column for the detection samples. (B) Analysis of THZ1 binding to DNA tetrahedron (TH)-4Z_{HER1} using 1% agarose gel electrophoresis. The gel was run at 110 V for 1 h, and stained with ethidium bromide. Lanes 1-5, 0.5 mM DNA tetrahedron (TH)-4Z_{HER1} incubated with 0-200 folds of THZ1.

FIGS. 4A-4H. Crosslinking of BC cells induced by I. MB-231 cells without drug treatment (A, D); with 500 nM THZ1 (B, E); with 10 nM I (C, F) were incubated 24 h and 48 h, respectively. (G) Calculated crosslinking efficiency of MB-231 cells in the presence of 10 nM I in 24 h (p<0.001); (H) Calculated crosslinking efficiency of BT474 cells in the presence of 10 nM I in 24 h.

FIGS. 5A-5E. Migration assays for MB231 breast cancer cells. (A) without drug treatment; (B) treatment with THZ1; (C) treatment with nanostructure I; (D) Migration rate of MB231 (migrated cell numbers/control cell numbers×100%) (p<0.001); (E) Invasion rate of MB231 (invaded cell numbers/control cell numbers×100%) (p<0.001).

FIGS. 6A-6B. Migration and invasion of BT474 cells. (A) Calculated migration rate of BT474 cells in the presence of 10 nM I (p>0.05); (B) Calculated invasion rate of BT474 cells in the presence of 10 nM I (p>0.05).

FIGS. 7A-7D. Aggregation of HCC1806 cells induced by nanostructures containing THZ1. (A) HCC1806 cells without drug treatment; (B) HCC1806 cells in the presence of 5 mM THZ1; (C) HCC1806 cells in the presence of 0.13 mM 4Z1-DNA tetrahedron-THZ1 nanostructure; (D) HCC1806 cells in the presence of 0.13 mM 4Z3-DNA tetrahedron-THZ1 nanostructure.

FIGS. 8A-8D. Aggregation of MDA-MB-231 cells induced by nanostructures containing doxorubicin (DOX). (A) MDA-MB-231 cells without drug treatment; (B) MDA-MB-231 cells in the presence of 5 mM DOX; (C) MDA-MB-231 cells in the presence of 0.13 mM 4Z1-DNA tetrahedron-DOX nanostructure; (D) MDA-MB-231 cells in the presence of 0.13 mM 4Z3-DNA tetrahedron-DOX nanostructure.

FIG. 9. Verification of linear DNA binding to THZ1. Double-strand linear DNAs were mixed with different amounts of THZ1 and loaded on 1% agarose gel. The gels were run at 150 V for 20 min, and stained with EtBr.

### DETAILED DESCRIPTION

The present invention has been described in terms of one or more preferred embodiments, and it should be appreciated that many equivalents, alternatives, variations, and modifications, aside from those expressly stated, are possible and within the scope of the invention.

The present disclosure provides tetrahedron nanostructures for targeting HER1 and HER3 positive cells. The nanostructures may be used to treat HER1 and HER3 positive cancers.

In a first aspect, provided herein is a nanostructure comprising a tetrahedron-shaped polynucleotide; and four peptides linked to the polynucleotide, wherein each of the peptides are selected from a HER1 binding peptide and a HER3 binding peptide. The peptides may comprise two or more HER1 binding peptides, two or more HER3 binding peptides, or a plurality of peptides including at least one HER1 peptide and at least one HER3 peptide. In exemplary embodiments, the tetrahedron nanostructure comprises four HER1 binding peptides, four HER3 binding peptides, or two HER1 binding peptides and two HER3 binding peptides. The HER1 and HER3 binding peptides may be located on the edges or the apexes of the tetrahedron, forming an asymmetric or symmetric structure.

As used herein, the term “nanostructure” refers to a chimeric structure comprising peptides and polynucleotides. The nanostructures described herein are tetrahedron-shaped, having six edges and four corners. The structure of the tetrahedron shape is formed by the polynucleotide, which may include up to six polynucleotide strands linked together. In exemplary embodiments, the nanostructure comprises four polynucleotide strands. Generally, DNA tetrahedron structures are described by Walsh et al., ACS Nano 5:5427-5432 (2011).

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

The polynucleotide comprising the tetrahedron nanostructure is preferably DNA, RNA, or a DNA or RNA derivative. The length of the polynucleotide can range from 10 to 1000 nucleotides.

DNA tetrahedrons may be comprised of single-stranded DNA polynucleotides. Single-stranded DNA polynucleotides that may be used in the tetrahedrons of the invention include: DNA₁ (5′—NH₂-AGG CAG TTG AGA CGA ACA TTC CTA AGT CTG AAA TTT ATC ACC CGC CAT AGT AGA CGT ATC ACC-3′ (SEQ ID NO:1)); DNA₂ (5′—NH₂-CCT CGC ATG ACT CAA CTG CCT GGT GAT ACG AGG ATG GGC ATG CTC TTC CCG ACG GTA TTG GAC-3′ (SEQ ID NO:2)); DNA₃ (5′—NH₂-CTT GCT ACA CGA TTC AGA CTT AGG AAT GTT CGA CAT GCG AGG GTC CAA TAC CGA CGA TTA CAG-3′ (SEQ ID NO:3)); DNA₄ (5′—NH₂-GGT GAT AAAACG TGT AGC AAG CTG TAA TCG ACG GGA AGA GCA TGC CCA TCC ACT ACT ATG GCG-3′ (SEQ ID NO:4)); DNA₉ (5′—NH₂-CAG TCT GAT TGC ATC GTT AGC TGT AGA TCG-3′ (SEQ ID NO:16)); and DNA₁₀ (5′—NH₂-CGA TCT ACA GCT AAC GAT GCA ATC AGA CTG-3′ (SEQ ID NO:5)). Other polynucleotide sequences suitable for use according to the peptide-DNA tetrahedron nanostructures described herein include: (5′—NH₂-CAA CTG CCT AGA CGA ACA TTC CTA AGT CTG AAA TTT ATC ACC CGC CAT AGT AGA CGT ATC ACC-3′ (SEQ ID NO:6)); (5′—NH₂-CCT CGC ATG ACT AGG CAG TTG GGT GAT ACG AGG ATG GGC ATG CTC TTC CCG ACG GTA TTG GAC-3′ (SEQ ID NO:7)); (5′-NH₂-TGT AGC AAG CGA TTC AGA CTT AGG AAT GTT CGA CAT GCG AGG GTC CAA TAC CGA CGA TTA CAG-3′ (SEQ ID NO:8)); and (5′—NH₂-GGT GAT AAA ACG CTT GCT ACA CTG TAA TCG ACG GGA AGA GCA TGC CCA TCC ACT ACT ATG GCG-3′ (SEQ ID NO: 9)).

The peptides may be linked to the polynucleotides using linkers. Suitable linkers include, without limitation, crosslinking agents having reactive moieties specific to various functional groups (e.g., sulfhydryls, amines, carbohydrates, azide, and alkyne). Exemplary linkers include, without limitation, N-[ε-maleimidocaproyloxy]succinimide ester; N-[ε-maleimidocaproyloxy]sulfosuccinimide ester; N-(β-Maleimidopropyloxy) succinimide ester; succinimidyl-4-[N-maleimidomethyl]cyclohexane-1-carboxylate; m-maleimidobenzoyl-N-hydroxysuccinimide ester.

The peptide may be linked to a 5′ amino-modifier of the polynucleotide using a linker. Embodiments incorporating 5′ linkage of the peptide to the polynucleotide are described in more detail in U.S. Pat. No. 10,532,109, which is a national phase application corresponding to PCT Publication No. 2017/200787, each of which are incorporated herein by reference.

Accordingly, the polynucleotides may comprise amino modifiers for linking the polynucleotides to the peptides. The polynucleotides may include a 5′ amino modifier, a 3′ amino modifier, or an internal amino modifier.

As used herein, “amino modification” or “amino modifier” refers to an amino modified nucleotide base or incorporation of primary amine modification into a polynucleotide without adding a base, such as by incorporation of an amino-modifier phosphoramidite backbone modification. The amino-modification in the polynucleotide may be an added amino modified base such as internal amino modifier C6 dT (iAmMC6T) or an amino-modifier phosphoramidite such as a Uni-Link™ modifier (iUniAmM), which incorporates the amino-modification without adding an additional nucleotide.

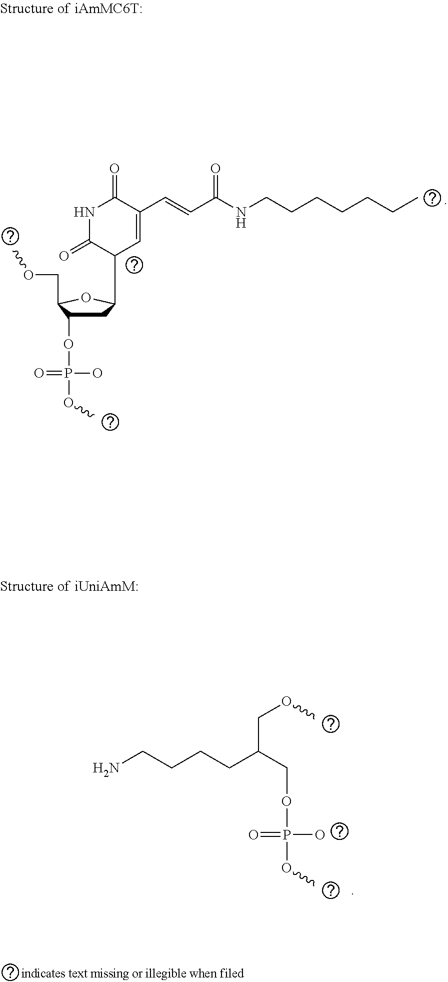

As used herein, “internal modification” or “internal amino-modification” or “internal amino modifier” refers to modification or amino-modification of a non-5′ terminal or non-3′ terminal nucleotide base in a polynucleotide, the modified nucleotide base being at least 1 nucleotide away from either the 3′ or 5′ terminus of the polynucleotide. For example, in the polynucleotide 5′-actgcta-3′ any of the nucleotides between and including the cytosine in the 2^{nd} position and the thymine in the 6^{th} position may be considered suitable for an internal modification. Modification of either the 5′ terminal adenine or the 3′ terminal adenine would not be considered an internal modification.

Suitable polynucleotides including amino modifiers include, but are not limited to DNA₅ (5′-AGG CAG TTG AGA CGA ACA TTC CTA AGT CTG AA/iAmMC6T/TTT ATC ACC CGC CAT AGT AGA CGT ATC ACC-3′ (SEQ ID NO:12)); DNA₆ (5′-CCT CGC ATG ACT CAA CTG CCT GGT GAT ACG/iAmMC6T/GG ATG GGC ATG CTC TTC CCG ACG GTA TTG GAC-3′ (SEQ ID NO:13)); DNA₇ (5′-CTT GCT ACA CGA TTC AGA CTT AGG AAT GTT CG/iAmMC6T/CAT GCG AGG GTC CAA TAC CGA CGA TTA CAG-3′ (SEQ ID NO: 14)); and DNA₈ (5′-GGT GAT AAAACG TGT AGC AAG CTG TAA TCG/iAmMC6T/CG GGA AGA GCA TGC CCA TCC ACT ACT ATG GCG-3′ (SEQ ID NO:15)). Other suitable sequences include any of SEQ ID NOs: 1-9 and 16, wherein any one of the nucleotides has been replaced with iAmMC6T. Additionally, the polynucleotide may have the sequence of any one of SEQ ID NOs: 1-9 and 12-16 wherein the sequence additionally includes iUniAmM between any two of the nucleotides. Suitable polynucleotides include sequences having at least 66%, 80%, 81%, 82%, 83%, 84%, 85%, 86%, 87%, 88%, 89%, 90%, 91%, 92%, 93%, 94%, 95%, 96%, 97%, 98% or 99% identity to any one of SEQ ID NOs: 1-9 and 12-16.

The peptides may be linked to the polynucleotide using a linker described herein linked to an internal amino modification of the polynucleotide. As used herein, “internal linkage” or “internally linked” refers to covalent or non-covalent attachment of the peptide or affibody to the polynucleotide at an internal amino-modified base or internal amino-modification or via formation of an imine linkage between a lysine sidechain residue and an aldehyde or ketone moiety engineered into the polynucleotide. Internal linkage is distinct from linkage of the peptide or affibody to a 5′ or 3′ amino-modified polynucleotide wherein the peptide or affibody is added to the end of the polynucleotide. For example, in the polynucleotide 5′-actgcta-3′, a covalent or non-covalent attachment to any of the nucleotides between and including the cytosine in the 2nd position and the thymine in the 6^{th} position is considered an internal linkage. Linkage at either the 5′ terminal adenine or the 3′ terminal adenine would be considered a terminal linkage and not an internal linkage.

The nanostructure may be made up of one, two, three, four, five, or six peptide-polynucleotide chimeras. A “peptide-polynucleotide chimera” is a molecule comprising a HER binding peptide, a linker, and a single stranded polynucleotide (e.g. a single stranded DNA molecule). Preferably, two or more peptide-polynucleotide chimeras are associated with each other to form a DNA nanostructure framework.

The terms “peptide,” “polypeptide,” and “protein” are used interchangeably to refer to a polymer of amino acid residues. The terms apply to amino acid polymers in which one or more amino acid residues is an artificial chemical analog of a corresponding naturally occurring amino acid, as well as to naturally occurring amino acid polymers. The terms polypeptide, peptide, and protein are also inclusive of modifications including, but not limited to, glycosylation, lipid attachment, sulfation, carboxylation, hydroxylation, ADP-ribosylation, and addition of other complex polysaccharides. The terms “residue” or “amino acid residue” or “amino acid” are used interchangeably to refer to an amino acid that is incorporated into a peptide, protein, or polypeptide. The amino acid may be a naturally occurring amino acid and, unless otherwise limited, may encompass non-natural analogues of natural amino acids that can function in a similar manner as naturally occurring amino acids.

The peptides described herein have a strong affinity for an extracellular domain of a member of the human epidermal growth factor receptor (HER) 1/epidermal growth factor receptor (EGFR) or HER3/EGFR3. The HER1 and HER3 binding peptides may range from 10 to 1000 amino acids in length. The HER1 and HER3 binding peptides may be affibodies. As used herein, the term “affibody” refers to a small, highly robust protein having high affinity for specific target proteins. Affibodies can be designed and used, for example, like aptamers. The HER1 and HER 3 affibodies may comprise three alpha helix bundle domains. The HER1 and HER 3 affibodies may have a molecular weight of approximately 6 kilodaltons (kDa). Suitable affibody sequences are known and available to practitioners in the art.

In exemplary embodiments, the HER1 binding peptide comprises the amino acid sequence of SEQ ID NO: 10 (MGHHHHHH LQVDNKFNKEMWAAWEEIRNLPNLNGWQMTAFIASLVDDPSQSANLLAEAKKLNDAQ APKVDC), or a sequence having at least 66%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO:10.

In exemplary embodiments, the HER3 binding comprises the amino acid sequence of SEQ ID NO: 11 (MGHHHHHH LQVDNKFNKERYLAYYEIWQLPNLNRTQKAAFIGSLQDDPSQSANLLAEAKKLNDAQA PKVDC), or a sequence having at least 66%, 80%, 81%, 82%, 83%, 84%, 85%, 86%, 87%, 88%, 89%, 90%, 91%, 92%, 93%, 94%, 95%, 96%, 97%, 98% or 99% identity thereto to SEQ ID NO: 11.

A polynucleotide or polypeptide has a certain percent “identity” or “similarity” to another polynucleotide or polypeptide, meaning that, when aligned, that percentage of bases or amino acids are the same, and in the same relative position, when comparing the two sequences. For example, the compared amino acid sequences are optimally aligned, and the number of amino acid differences are counted and converted to a percentage. For example, if a first amino acid sequence of 50 amino acids is optimally aligned with a second amino acid sequence of 50 amino acids, and 5 out of 50 amino acids differ from the second amino acid sequence, then the first amino acid sequence is said to have 10% identity with the second amino acid sequence. Sequence identity can be determined in a number of different manners, such as alignment using computer programs, including alignment algorithms such as BLAST (available on the World Wide Web at ncbi.nlm.nih.gov/BLAST) and FASTA (available in the Genetics Computing Group (GCG) package).

An exemplary protocol for synthesizing a tetrahedron nanostructure comprises reacting four peptide-polynucleotide chimeras (e.g., affibody-polynucleotide chimeras) in the presence of 10 mM Tris. HCl, pH 8.0, containing 10 mM of MgCl₂, and incubating the reaction mixture at about 60 °C to about 90 °C. These protocols are described in PCT Publication No. 2021/021523, which is incorporated herein by reference.

The nanostructure described herein may further comprise a plurality of molecules of at least one drug. The drug may comprise a small molecule drug. As used herein a “small molecule drug” is a low molecular weight (≤1000 dalton) organic compound.

Small molecule drugs for inclusion in the nanostructures described herein include, without limitation, THZ1, doxorubicin (DOX), epirubicin, docetaxel, ribociclib, daunorubicin, etoposide, camptothecin, cisplatin, mitomycin C, bleomycin, cyclophosphamide, 5-fluorouracil, hydroxyurea, cytosine arabinoside, and gemcitabine. The small molecule drug may be a CDK7 inhibitor. CDK inhibitors for inclusion in a polynucleotide tetrahedron nanostructure described herein include, without limitation THZ2, Alvocidib, Seliciclib, SNS-032. Other useful CDK inhibitors include those described in Sava et al., Cancer and Metastasis Reviews 39:805-823 (2020).

The drugs may be covalently or non-covalently bound to the nanostructure. The nanostructures described herein provide a highly efficient approach to specifically delivering small molecule drugs to HER1 and HER3 overexpressed cancer cells. When a plurality of molecules of a drug are bound to a nanostructure as described herein via non-covalent binding, the plurality of molecules can dissociate from the nanostructure upon entry or contact to the target cancer cell.

Each vehicle nanostructure has capacity to delivery tens to hundreds of molecules of an anti-cancer drug. A nanostructure may comprise between 5 and 1000 molecules of the drug or any number or range in between, e.g. 5, 10, 20, 30, 40, 50, 60, 70, 80, 90, 100, 200, 300, 400, 500, 600, 700, 800, 900, or 1,000 molecules. The nanostructure may comprise between 10 and 50 molecules of the drug.

The drug may be attached to the nanostructure using, for example, a degradable linker. Linkers suitable for attaching the drug to the nanostructure include, without limitation, DNA, RNA, peptides, polysaccharides, esters, amides, and disulfide bonds.

In a second aspect, provided herein is a pharmaceutical composition comprising the nanostructure described herein and a pharmaceutically acceptable carrier.

The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition.

In a third aspect, provided herein are methods for treating cancers associated with overexpression of at least one of HER1 and HER3 molecules. The cancer may be triple negative breast cancer. As used herein, the terms “treating,” “treat,” and “treatment” refer to the management and care of a subject or patient for the purpose of combating the disease, condition, or disorder. The terms embrace both preventative, i.e., prophylactic, and palliative treatments. In some cases, the term “treated” refers to any beneficial effect on progression of a disease or condition. Beneficial effects can include reversing, alleviating, inhibiting the progress of, preventing, or reducing the likelihood of the disease or condition to which the term applies or one or more symptoms or manifestations of such a disease or condition. Where the disease or condition is a tumor, cancer, or cancer-associated condition, treating can refer to the management and care of a patient for the purpose of combating cancer, and can include reversing, alleviating, inhibiting the progress of, preventing, reducing the size of, or reducing the likelihood of, or lessening the severity of any aspect of the cancer or cancer-associated condition (e.g., metastasis, tumor growth). A therapeutic beneficial effect on the health and well-being of a patient includes, but it not limited to: (1) curing the cancer; (2) slowing the progress of the cancer; (3) causing the tumor to regress; or (4) alleviating one or more symptoms of the cancer. As used herein, the terms “preventing” and “prevent” refer not only to a complete prevention of a certain disease or condition, but also to partially or substantially attenuating, reducing the risk of, or delaying the development or recurrence of the disease or condition to which the term applies.

The cancer may be a late-stage cancer. As used herein, the term “late-stage cancer” refers a clinical stage III or stage IV cancer as determined by a qualitative or quantitative assessment of the level of advancement of a cancer. Criteria used to determine the stage of a cancer include, without limitation, the size of the tumor, amount of nodes where the cancer has spread, and the extent of metastases (e.g., localized or distant), and will be known to those skilled in the art. It will be understood by those skilled in the art that criteria for determining the stage of a cancer may vary depending on the type of cancer.

The methods directed to treating or preventing a cancer in a subject comprise administering a therapeutically effective amount of a nanostructure or pharmaceutical composition provided herein. A “therapeutically effective amount” refers to an amount of the nanostructure that alleviates, in whole or in part, symptoms associated with a disorder or disease, or slows or halts further progression or worsening of those symptoms, or prevents or provides prophylaxis for the disease or disorder in a subject at risk for developing the disease or disorder. A therapeutically effective dose relates to the amount of a treatment that is sufficient to improve the symptoms, for example a healing, prevention or improvement of such conditions. For dosage determinations, it can be advantageous to assess toxicity and therapeutic efficacy of a compound in cell cultures or in experimental animals. For example, the LD50 (i.e., the dose lethal to 50% of the population) and

ED50 (i.e., the dose therapeutically effective in 50% of the population) can be determined. From these calculations, dosage ranges for use in humans can be formulated. Dosage ranges can vary depending on factors such as mode of administration. A therapeutically effective amount of a pharmaceutical composition provided herein can range from about 0.001 to 100 mg of nanostructure per kg body weight of the subject (e.g., about 0.01 to 100 mg/kg body weight; about 0.1 to 40 mg/kg body weight; about 1 to 20 mg/kg body weight).

A “subject” or “patient” is a member of any animal species, preferably a mammalian species, optionally a human. The subject can be an apparently healthy individual, an individual suffering from a disease, or an individual being treated for a disease.

The terms “administering,” “administer,” “delivering,” and “deliver,” can be used interchangeably to indicate the introduction of any agent (e.g., a therapeutic agent) into the body of a patient in need thereof to treat a disease or condition, and can further mean the introduction of any agent into the body for any purpose. The terms encompass any route of introducing or delivering to a subject a compound to perform its intended function. A composition comprising a nanostructure as provided herein can be delivered or administered to a subject by any method that achieves the intended purpose or is deemed appropriate by those of skill in the art. For example, a composition of the present invention can be administered as a pharmaceutical, and may be administered systemically or locally via oral or parenteral administration. Oral administration includes, for example, administration of oral agents. Such oral agents include, for example, granules, powders, tablets, capsules, solutions, emulsions, and suspensions. Parenteral administration includes, for example, administration of injections. Such injections include, for example, subcutaneous injections, intramuscular injections, and intraperitoneal injection. Intravenous injections such as drip infusions, intramuscular injections, intraperitoneal injections, subcutaneous injections, suppositories, enemas, oral enteric tablets, or the like can be selected. The nanostructure may be administered with a drug delivery device. “Drug delivery device” encompasses any and all devices that administers a therapeutic agent to a patient and includes infusion pumps, implanted or percutaneous vascular access ports, direct delivery catheter systems, local drug-release devices or any other type of medical device that can be adapted to deliver a therapeutic to a patient.

Appropriate modes of administration can be determined based on the physical location of a tumor or tumors in the subject's body. In exemplary embodiments, a composition comprising a nanostructure as provided herein is administered to a subject having a diagnosis of breast cancer or a pre-cancerous lesion, where the composition is administered orally or intravenously. Alternatively, a composition comprising a nanostructure can be administered locally to an intended area of treatment. For example, a composition comprising the nanostructure can be administered by local application during surgery.

Compositions can be administered to a subject in need thereof in dosage unit form where each discrete dosage unit contains a predetermined quantity of an active ingredient or compound that was calculated to elicit a desirable therapeutic effect when administered with, in some cases, a pharmaceutically acceptable buffer or carrier. The terms “pharmaceutically acceptable buffer” and “pharmaceutically acceptable carrier” are meant to encompass any buffer or carrier, which does not interfere with the effectiveness of the biological activity of the active ingredient and that is not toxic to the host to which it is administered.

Treatment or prevention according to a method provided herein can occur before, during, or after the subject is treated by surgery, radiation, and/or chemotherapy. In some cases, treatment according to a method provided herein prior to chemo- or radiotherapy may improve the outcome of the conventional therapy. A nanostructure as provided herein may be administered to a subject concurrently with one or more other treatments or preventative measures such as radiotherapy, chemotherapy, or surgery.

In a fourth aspect, provided herein are methods for crosslinking cells overexpressing at least one of HER1 and HER3 molecules comprising treating the cells with the nanostructure disclosed herein. As used herein, “crosslinking” refers to binding at least two cancer cells together. The nanostructures described herein may serve as a linker between the two cells.

In other aspects, the nanostructure can be another structure, such as linear type, Y-type, or polygon structure, or any other geometric structure recognized as suitable by persons of ordinary skill in the art in view of the teachings herein. Other nanostructure shapes contemplated are disclosed in WO2017200787A1, which is incorporated herein by reference in its entirety.

The indefinite articles “a” and “an,” as used herein, unless clearly indicated to the contrary, should be understood to mean “at least one.”

A range includes each individual member. Thus, for example, a group having 1-3 members refers to groups having 1, 2, or 3 members.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Unless defined otherwise, all technical and scientific terms used herein have the same meaning as commonly understood by one of skill in the art to which the invention pertains. All definitions, as defined and used herein, should be understood to control over dictionary definitions, definitions in documents incorporated by reference, and/or ordinary meanings of the defined terms.

### Examples

Reference is now made to the following examples, which together with the above descriptions illustrate some embodiments of the invention in a non-limiting fashion.

### Example 1

Breast cancers are divided into four major subgroups: luminal A, luminal B, HER2 overexpressing, and basal-like (ER-/PR-/HER2—), according to the expression of two hormone receptors, estrogen receptor (ER) and progesterone receptor (PR), and a transmembrane receptor, human epidermal growth factor receptor 2 (HER2).^{1,2} The basal-like subgroup is also commonly called triple-negative breast cancer (TNBC) defined by negative expression of ER, PR, and HER2. Among all subgroups, TNBC and HER2-overexpressing breast cancer have higher metastasis rates and worse prognoses.³⁻⁵ Metastasis of breast cancer is the major cause of death from this disease. TNBC is highly invasive, and approximately 46% of TNBC patients will have distant metastasis. Chemotherapy is the main systemic treatment for TNBC. Therefore, there is an urgent need to develop new agents to specifically target TNBC to inhibit its growth and metastasis.

Most TNBC cells are HER1 and/or HER3 positive.⁶⁻⁹ Expression of the HER1 and HER3 is associated with tumor recurrence and more aggressive features. HER1 (also known as EGFR) is a member of a transmembrane receptor family, which in humans includes four epidermal growth factor receptors (HER1/EGFR, HER2, HER3, and HER4).²¹ Inappropriate activation of these HER receptors is associated with the development, metastasis, and therapeutic resistance of many cancers.²²⁻²⁷ Recently, several antibodies and antibody-drug conjugates were under development for TNBC-targeting therapy.²⁸⁻³⁰ Although clinical trials have not yielded positive results, the HER1-targeting approach is still of particular interest since HER1 overexpression potentiates key functions of cancer cells.³¹⁻³³ Several studies have confirmed that HER1 is an important target in TNBC, in which at least 50% of cases overexpress HER1.^{6,7} Anti-HER1 monoclonal antibodies (cetuximab and panitumumab) have been approved by the FDA to treat metastatic colorectal cancer, metastatic non-small cell lung cancer, and head and neck cancer.^{10,11} However, neither antibody confers significant benefit to TNBC patients when employed as a single agent.⁶ Nevertheless, HER1-targeting antibodies can enhance the chemosensitivity of TNBC cells in TNBC patients.^{28,29}

The HER3 overexpression rate is greater than 41% in TNBC.⁹ There is a significant correlation between HER3 overexpression and a high rate of metastasis as well as low rate of survival in TNBC patients.⁹ Recently, HER3 has gained more attention as a therapeutic target TNBC. Patritumab deruxtecan (U3-1402) is a HER3 antibody drug conjugate, which has demonstrated early and clinically meaningful activity for breast cancer patients.¹²

While current therapeutic drug development for cancer treatment focuses on binding to one intracellular or extracellular target for inhibiting the growth of cancer cells, no current therapy targets the intercellular communication of cancer cells to block their growth and metastasis.

Several series of affibody molecules (Z_{HER1}, Z_{HER2}, and Z_{HER3}) that encompass only 58 amino acids (6 kDa) have been reported.³⁴⁻³⁷ Affibody molecules are derived from human immunoglobulin G protein Z-domain scaffolds. Each affibody contains three α-helical bundles, each of which specifically targets the extracellular domain of a HER receptor (HER1, HER2, or HER3).³⁴ These affibody molecules can be expressed in E. coli BL21 (DE3) cells at a high level, and prepared at a much lower cost than monoclonal antibodies. These affibody molecules have similar high affinities to their HER receptors as monoclonal antibodies, but are not naturally present in human cells. Of note, none of these affibody molecules has an inhibitory effect on cancer cell growth, even if they have strong selective affinities to their receptors.³⁸

THZ1 is a newly discovered TNBC inhibitor. In TNBC, one cyclin-dependent kinase (CDK), called CDK7, was found to play an essential role in gene transcription initiation and elongation.^{39,40} The growth of TNBC cells is dependent on the activity of CDK7. Recently, a newly discovered covalent CDK7 inhibitor, THZ1, has been reported to selectively inhibit the growth of TNBC cells (IC₅₀<100 nM).^{39,40}

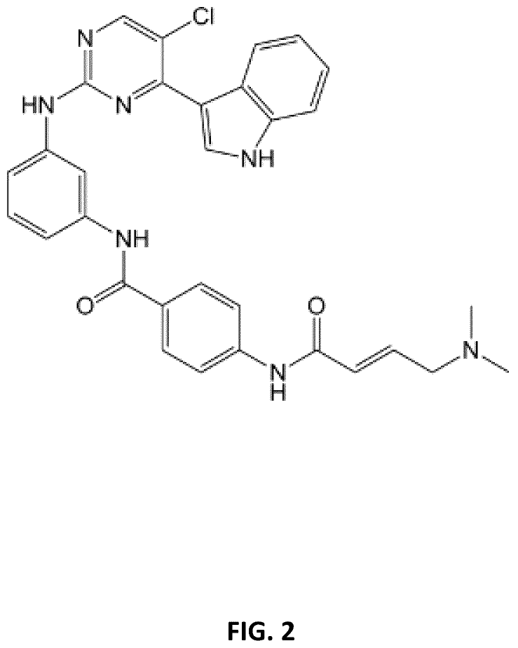

We prepared a novel DNA tetrahedron-4Z_{HER1}-THZ1 nanostructure (I), which contains four HER1 specific affibody molecules (Z_{HER1}) and ten-fold THZ1. The structure of THZ1 is shown in FIG. 2.

### Preparation of DNA-Z_{HER1}

TABLE 1
DNA sequences for preparing DNA
4Z_{HER1}-THZ1 nanostructure.

| Name | Sequences |
|---|---|
| DNA₅ | 5′-AGG CAG TTG AGA CGA ACA |
| | TTC CTA AGT CTG AA/iAmMC6T/ |
| | TTT ATC ACC CGC CAT AGT AGA |
| | CGT ATC ACC-3′ (SEQ ID NO: 12) |
| DNA₆ | 5′-CCT CGC ATG ACT CAA CTG |
| | CCT GGT GAT ACG /iAmMC6T/GG |
| | ATG GGC ATG CTC TTC CCG ACG |
| | GTA TTG GAC-3′ (SEQ ID NO: 13) |
| DNA₇ | 5′-CTT GCT ACA CGA TTC AGA |
| | CTT AGG AAT GTT CG/iAmMC6T/ CAT |
| | GCG AGG GTC CAA TAC CGA CGA |
| | TTA CAG-3′ (SEQ ID NO: 14) |
| DNA₈ | 5′-GGT GAT AAAACG TGT AGC AAG |
| | CTG TAA TCG /iAmMC6T/CG |
| | GGA AGA GCA TGC CCA TCC ACT |
| | ACT ATG GCG-3′ (SEQ ID NO: 15) |

The sequences of the single-strand DNAs used for preparing the DNA tetrahedrons are listed in Table 1. The Z_{HER1} affibody was expressed in E. coli cells and purified using a Ni-NTA column.

Each single-strand DNA (200 μg, 10.3 nmol) was dissolved in 160 μL of phosphate-buffered saline (PBS, 10 mM PO₄³⁻, 137 mM NaCl, and 2.7 mM KCl) and treated with 40 μL of 10 mM N^{ε}-malemidocaproyloxysuccinimide ester (EMCS) in dimethyl sulfoxide. The reaction mixture was incubated at room temperature overnight and stopped by the addition of 20 μL of 3 M NaOAc. After the addition of 600 μL of ethanol and incubation at 4 °C for 30 min, the reaction mixture was centrifuged at 15000×g for 30 min. After washing with 70% ethanol, the DNA was dissolved in 50 μL of PBS buffer and treated with 300 μg (38.1 nmol) of affibody in 300 μL of PBS buffer. After incubation at room temperature for 1 h, the reaction mixture was stopped by the addition of 35 μL of 3M NaOAc. After the addition of 1000 μL of ethanol and incubation at 4 °C for 30 min, the reaction mixture was centrifuged at 15000×g for 30 min. After washing with 70% ethanol, the DNA-affibody was dissolved in 200 μL of 50 mM Tris-HCl, pH 8.0, containing 300 mM NaCl.

The DNA-affibody in the solution was purified on a Ni-NTA chromatography column. The crude product solution (200 μL) was loaded on a column containing 300 μL of Ni-NTA resin. Then the column was washed five times with 300 μL of 50 mM Tris-HCl, pH 8.0, containing 300 mM NaCl and 5 mM imidazole. Finally, the Ni-NTA column was eluted three times with 300 μL of 50 mM Tris-HCl, pH 8.0, containing 30 mM NaCl and 150 mM imidazole. The eluate from the previous step was purified further on a column containing 200 μL DEAE-Sepharose. The column was eluted with 200 μL PBS buffer containing 0.2-0.9 M NaCl. To each eluate was added 20 μL of 3 M NaOAc and 600 μL of ethanol. After incubation at 4 °C for 30 min, the mixture was centrifuged at 15000×g for 30 min. After washing with 70% ethanol, the product was dissolved in 200 μL of H₂O. The product was analyzed by 8% denaturing polyacrylamide gel electrophoresis (PAGE). The gel was run at 110 V for 1 h, and stained with ethidium bromide.

### Preparation of DNA Tetrahedron-Z_{HER1} Nanostructure

DNA₅-Z_{HER1} (10.0 nmol), DNA₆-Z_{HER1} (10.0 nmol), DNA₇-Z_{HER1} (10.0 nmol), and DNA₈-Z_{HER1} (10.0 nmol) were added to 8 mL of 10 mM Tris. HCl, pH 8.0, containing 10 mM MgCl₂. The reaction mixture was incubated at 85 °C for 10 min then cooled to room temperature over a period of 30 min. The obtained DNA tetrahedron-affibody nanostructures were added 100 nmol THZ1.

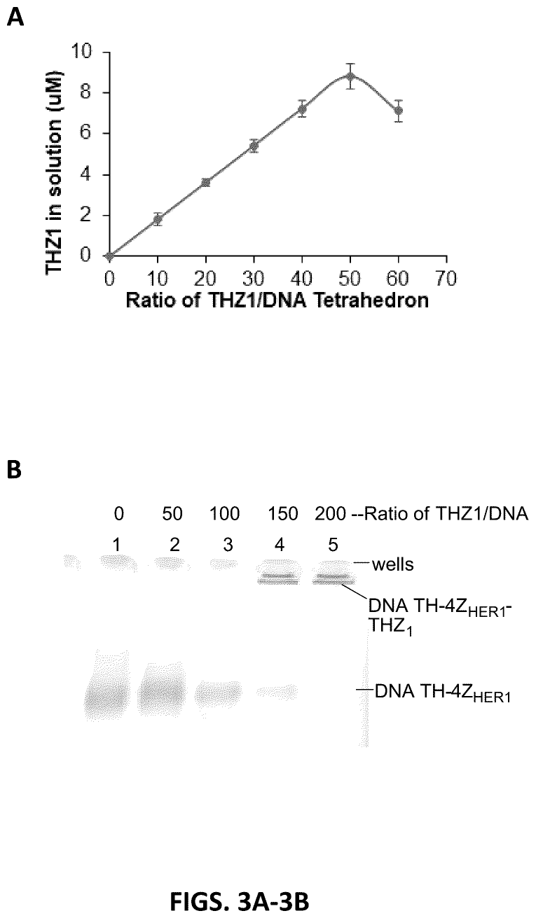

DNA-THZ1 binding assay: The DNA binding property of THZ1 was evaluated following our reported method.⁴³ DNA tetrahedron was incubated with 10-60 fold of THZ1. After purification with G-25 column to remove unbound THZ1, the amount of THZ1 in the DNA tetrahedron was measured using UV absorption at 220 nm. As shown in FIG. 3A, the concentration of THZ1 increased linearly when the ratio of THZ1/DNA tetrahedron increased from 10 to 50. However, when the ratio was 60, part of THZ1/DNA complex did not pass through the G-25 column. Then we used a 1% agarose gel to analyze the binding property for high concentrations of THZ1. As shown in FIG. 3B, when the ratio of THZ1/DNA was lower than 50, the electrophoretic mobility of DNA tetrahedron-4Z_{HER1}-THZ1 nanostructure (I) was not affected. However, when the ratio was higher than 50, more THZ1 accumulated to the DNA tetrahedron (TH)-4Z_{HER1} nanostructure; resulted in DNA structure changes; and then dramatically decreased its electrophoretic mobility. Thus, we used 50-fold of THZ1 to prepare DNA tetrahedron-4Z_{HER1}-THZ1 nanostructure (I) for following assays.

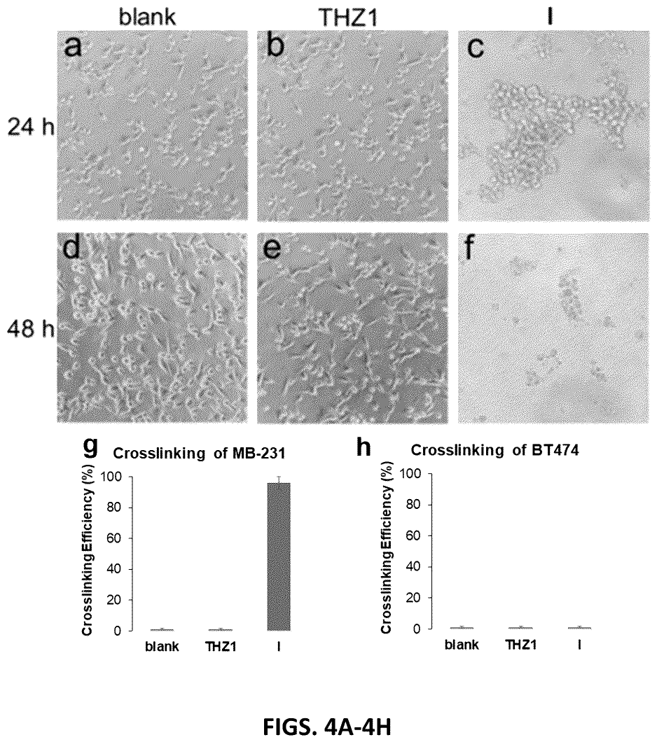

Crosslinking assay: Crosslinking of TNBC cells by I was verified using a HER1 overexpressing TNBC cell line (MDA-MB-23146). In the presence of nanostructure I (containing 50 copies of THZ1), the cells were effectively crosslinked to each other through multiple Z_{HER1}-HER1 bindings. As shown in FIG. 4, untreated cells and cells treated with 500 nM free THZ1 were not aggregated after incubation for 24 h (FIGS. 4A and 4B). In comparison, treatment with 10 nM nanostructures I (10 nM), which contains four affibodies oriented in a fashion that enables binding to two adjacent cells; resulted in significant cell aggregation (FIG. 4C). The crosslinking efficiency was up to 96% in 24 h (FIG. 4G). [Crosslinking efficiency (%)=(cell number without any treatment-non-aggregated cell number)× 100%/(cell number without any treatment). Data were reported as the mean of three independent experiments]. As a negative control, HER1 negative BT474 BC cells were not aggregated under the same conditions (FIG. 4H). After 48 h incubation, untreated cancer cells kept growing (FIG. 4D); and THZ1 treated cells were partially growth inhibited (FIG. 4E). However, all MB-231 cells died after 48 h in the presence of 10 nM nanostructure I (FIG. 4F). Therefore, drug-loaded nanostructure I has the ability to crosslink the TNBC cells and enhance apoptosis compared to corresponding free drug.

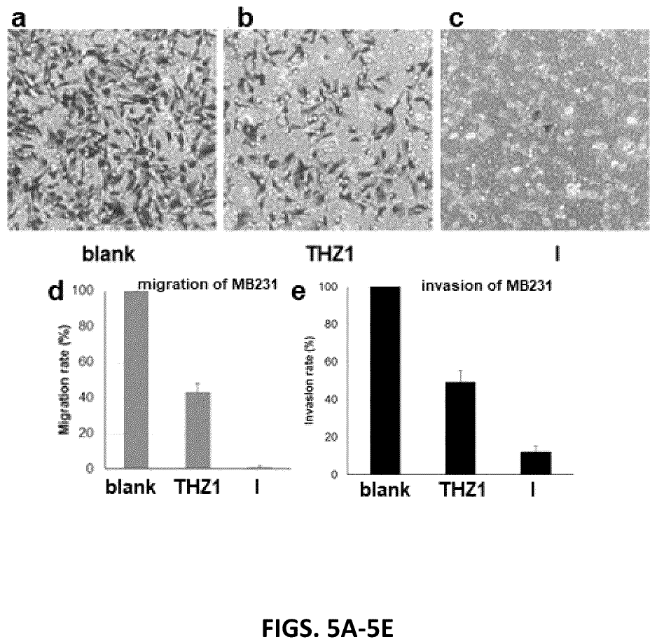

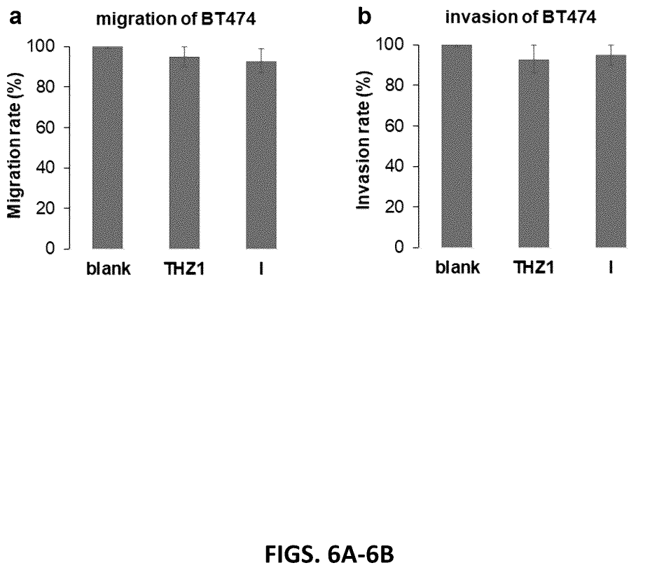

Migration assay: To verify whether nanostructure I blocks TNBC cell dissociation from aggregated cell clusters, a Transwell™ migration assay was performed. As shown in FIGS. 5B and 5D, THZ1 inhibited the migration of MB231 cancer cells to the extent of 57% at 500 nM concentration in 24 h, probably due in part to its strong inhibition of TNBC cell growth.^{39,40} Comparatively, 10 nM nanostructure I (containing 500 nM THZ1) inhibited the migration of MB231 cancer cells to the extent of 99% (FIGS. 6C and 6D) in 24 h. Since nanostructure I contains the same amount of THZ1, the enhanced inhibition of migration by nanostructure I is attributed to its ability to crosslink the cells. As a negative control, the migration of HER1 negative BT474 BC cells were not inhibited by I (FIG. 6A).

Invasion assay: To verify whether nanostructure I blocks cells from passing through vessel walls, an invasion assay was performed using a matrigel matrix treated Transwell™ to mimic the vessel wall. As shown in FIG. 5E, THZ1 inhibited the invasion of MB231 cancer cells to the extent of 51% at 500 nM concentration, but nanostructure I inhibited invasion to the extent of 90% at 10 nM concentration (containing the same amount of THZ1) in 24 h. As a negative control, the invasion of HER1 negative BT474 BC cells were not affected by I (FIG. 6B).

In summary, nanostructure I containing four Z_{HER1} affibody molecules at its four apexes, specifically targets HER1 receptor, exhibits higher selectivity to HER1 than free THZ1, resulting in increased MB231 cell aggregation and marked reduction in cell migration and invasion.

### Example 2

The aggregation effects of drug-loaded DNA-4Z_{HER1/3}-nanostructures were further studied.

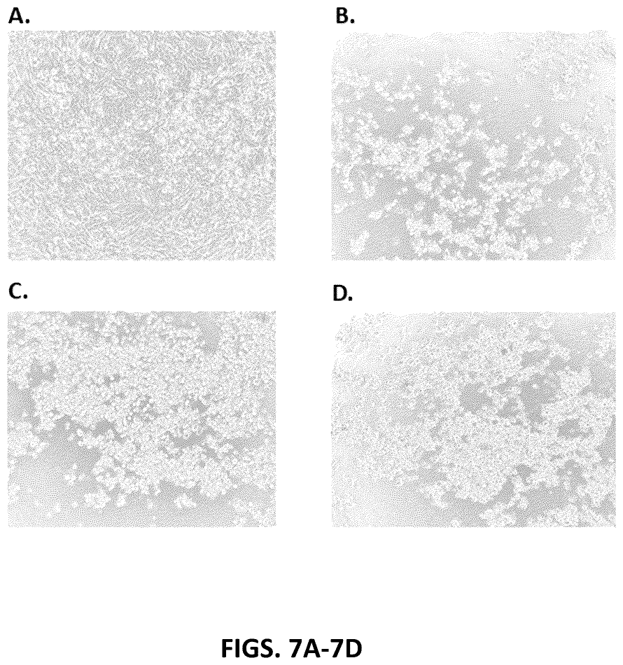

The effect of THZ1-loaded DNA-4Z_{HER1} nanostructures and DNA-4Z_{HER3} nanostructures on aggregation of HCC1806 cells was evaluated (FIGS. 7A-7D). The DNA-4Z_{HER3} nanostructures were prepared similarly as discussed above for DNA-4Z_{HER1}, using DNA₅, DNA₆, DNA₇, and DNA₈.

To evaluate the ability of the THZ1-loaded DNA-4Z_{HER1} and DNA-4Z_{HER3} nanoparticles to crosslink HER1 or HER3 overexpressing cancer cells, HCC1806 breast cancer cells (HER 1/HER3 overexpression) were cultured at 37 °C for 24 h. (A) HCC1806 cells without drug treatment; (B) HCC1806 cells in the presence of 5 μM THZ1; (C) HCC1806 cells in the presence of 0.13 μM 4Z1-DNA tetrahedron-THZ1 nanoparticle; (D) HCC1806 cells in the presence of 0.13 μM 4Z3-DNA tetrahedron-THZ1 nanoparticle. Results show that the DNA-4Z_{HER1} and DNA-4Z_{HER3} nanoparticles containing THZ1 can crosslink the HCC1806 cells.

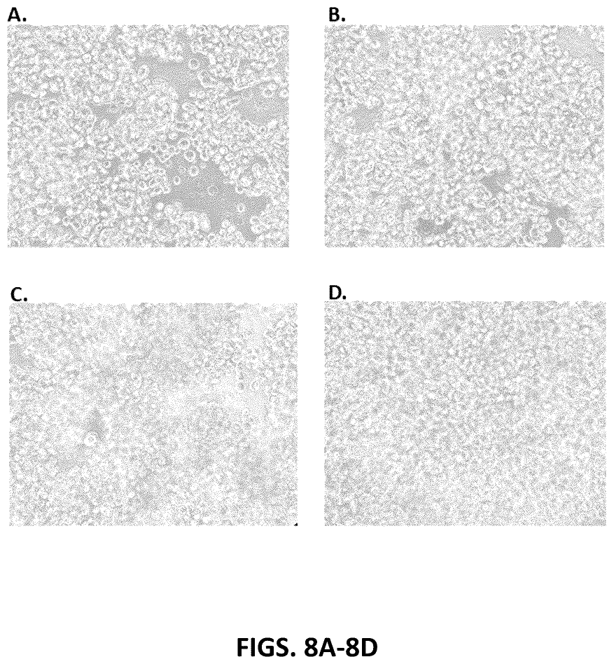

The effect of DOX-loaded DNA-4Z_{HER1} nanostructures and DNA-4Z_{HER3} nanostructures on aggregation of MDA-MB-231 cells was evaluated (FIGS. 8A-8D). To evaluate the ability of the DOX-loaded DNA-4Z_{HER1} and DNA-4Z_{HER3} nanoparticles to crosslink HER1 or HER3 overexpressing cancer cells, MDA-MB-231 breast cancer cells (HER1 overexpression; HER3 low expression) were cultured at 37 °C for 24 h. (A) MDA-MB-231 cells without drug treatment; (B) MDA-MB-231 cells in the presence of 5 μM DOX; (C) MDA-MB-231 cells in the presence of 0.13 μM 4Z1-DNA tetrahedron-DOX nanoparticle; (D) MDA-MB-231 cells in the presence of 0.13 M4Z3-DNA tetrahedron-DOX nanoparticle. Results show that the DNA-4Z_{HER1} and DNA-4Z_{HER3} nanoparticles containing DOX can crosslink the MDA-MB-231 cells. Comparatively, DOX-DNA-4Z_{HER1} results in more crosslinking than DOX-DNA-4Z_{HER3}.

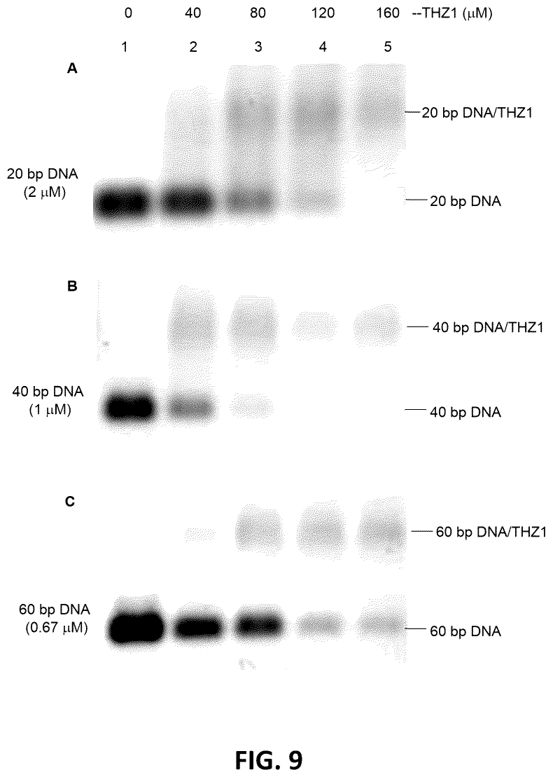

Binding of DNA to THZ1 was evaluated (FIG. 9). Double-strand linear DNAs (20 bp, 40 bp and 60 bp) were prepared by mixing two complimentary DNAs (as listed below) in 50 mM Tris-HCl, pH 7.5, containing 50 mM NaCl. The reaction mixtures were heated at 90 °C for 5 min, then cool to room temperature. To keep the same base-pair number for each same, the 20 bp DNA was adjusted to 2 mM; 40 bp DNA was adjusted to 1 mM; 60 bp DNA was adjusted to 0.67 mM. Then 40-160 mM THZ1 were added to each sample and loaded on 1% agarose gel. The results show that THZ1 can bind to double-strand DNA.

### DNA Sequences for Linear Double-Strand DNAs of FIG. 9.

20-nt-F
(SEQ ID NO: 17)
TAG ATC AGT CTG TAA GCG CC
20-nt-R
(SEQ ID NO: 18)
GGC GCT TAC AGA CTG ATC TA
40-nt-F
(SEQ ID NO: 19)
TAG ATC AGT CTG TAA GCG CCG
TTA GCG GTA TAG CCC GTT A
40-nt-R
(SEQ ID NO: 20)
TAA CGG GCT ATA CCG CTA ACG
GCG CTT ACA GAC TGA TCT A
60-nt-F
(SEQ ID NO: 21)
TAG ATC AGT CTG TAA GCG CCG
TTA GCG GTA TAG CCC GTT ATC
GGC TAG GAA AAC GCC TAG
60-nt-R
(SEQ ID NO: 22)
CTA GGC GTT TTC CTA GCC GAT
AAC GGG CTA TAC CGC TAA CGG
CGC TTA CAG ACT GAT CTA

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

We claim:

1. A nanostructure comprising:
a tetrahedron-shaped polynucleotide; and
four peptides linked to the polynucleotide,
wherein each of the peptides are selected from a HER1 binding peptide and a HER3 binding peptide.

2. The nanostructure of claim 1, wherein the nanostructure comprises four HER1 binding peptides.

3. The nanostructure of claim 1, wherein the nanostructure comprises four HER3 binding peptides.

4. The nanostructure of claim 1, wherein the tetrahedron-shaped polynucleotide comprises four polynucleotide strands, wherein the sequences of the four polynucleotide strands individually comprise SEQ ID NOs: 12, 13, 14, and 15.

5. The nanostructure of claim 1, wherein the HER1 binding peptide comprises SEQ ID NO: 10, or a sequence having at least 95% identity thereto.

6. The nanostructure of claim 1, wherein the HER3 binding peptide comprises SEQ ID NO: 11, or a sequence having at least 95% identity thereto.

7. The nanostructure of claim 1, wherein each of the peptides are affibodies.

8. The nanostructure of claim 1, wherein the polynucleotide is DNA or RNA.

9. The nanostructure of claim 1, further comprising a plurality of molecules of at least one drug.

10. The nanostructure of claim 9, wherein the drug is a CDK7 inhibitor.

11. The nanostructure of claim 10, wherein the drug is THZ1.

12. The nanostructure of claim 11, wherein the nanostructure comprises between 5 and 1,000 molecules of THZ1.

13. The nanostructure of claim 12, wherein the nanostructure comprises between 10 and 50 molecules of THZ1.

14. The nanostructure of claim 9, wherein the at least one drug is selected from the group consisting of doxorubicin, epirubicin, docetaxel, ribociclib, THZ2, Alvocidib, Seliciclib, and SNS-032.

15. A pharmaceutical composition comprising the nanostructure of claim 1 and a pharmaceutically acceptable carrier.

16. A method of treating a cancer associated with overexpression of at least one of HER1 and HER3 in a subject in need thereof, the method comprising administering to the subject a therapeutically effective amount of the pharmaceutical composition of claim 15.

17. The method of claim 16, wherein treating the cancer comprises reducing metastasis of the cancer.

18. The method of claim 16, wherein treating the cancer comprises reducing growth of the cancer.

19. The method of claim 16, wherein the cancer is triple negative breast cancer.

20. A method of crosslinking cancer cells overexpressing at least one of HER1 and HER3, the method comprising treating the cells with the nanostructure of claim 1.


SEQUENCE LISTING (22 sequences)

SEQ ID NO: 1 - DNA, 63 bp, synthetic construct
  source 1..63: mol_type = other DNA
aggcagttgagacgaacattcctaagtctgaaatttatcacccgccatagtagacgtatcacc

SEQ ID NO: 2 - DNA, 63 bp, synthetic construct
  source 1..63: mol_type = other DNA
cctcgcatgactcaactgcctggtgatacgaggatgggcatgctcttcccgacggtattggac

SEQ ID NO: 3 - DNA, 63 bp, synthetic construct
  source 1..63: mol_type = other DNA
cttgctacacgattcagacttaggaatgttcgacatgcgagggtccaataccgacgattacag

SEQ ID NO: 4 - DNA, 63 bp, synthetic construct
  source 1..63: mol_type = other DNA
ggtgataaaacgtgtagcaagctgtaatcgacgggaagagcatgcccatccactactatggcg

SEQ ID NO: 5 - DNA, 30 bp, synthetic construct
  source 1..30: mol_type = other DNA
cgatctacagctaacgatgcaatcagactg

SEQ ID NO: 6 - DNA, 63 bp, synthetic construct
  source 1..63: mol_type = other DNA
caactgcctagacgaacattcctaagtctgaaatttatcacccgccatagtagacgtatcacc

SEQ ID NO: 7 - DNA, 63 bp, synthetic construct
  source 1..63: mol_type = other DNA
cctcgcatgactaggcagttgggtgatacgaggatgggcatgctcttcccgacggtattggac

SEQ ID NO: 8 - DNA, 63 bp, synthetic construct
  source 1..63: mol_type = other DNA
tgtagcaagcgattcagacttaggaatgttcgacatgcgagggtccaataccgacgattacag

SEQ ID NO: 9 - DNA, 63 bp, synthetic construct
  source 1..63: mol_type = other DNA
ggtgataaaacgcttgctacactgtaatcgacgggaagagcatgcccatccactactatggcg

SEQ ID NO: 10 - protein, 71 aa, synthetic construct
MGHHHHHHLQVDNKFNKEMWAAWEEIRNLPNLNGWQMTAFIASLVDDPSQSANLLAEAKKLNDAQAPKVDC

SEQ ID NO: 11 - protein, 71 aa, synthetic construct
MGHHHHHHLQVDNKFNKERYLAYYEIWQLPNLNRTQKAAFIGSLQDDPSQSANLLAEAKKLNDAQAPKVDC

SEQ ID NO: 12 - DNA, 63 bp, synthetic construct
  source 1..63: mol_type = other DNA
  modified_base 33: mod_base = OTHER; note = n is amino modified base
aggcagttgagacgaacattcctaagtctgaantttatcacccgccatagtagacgtatcacc

SEQ ID NO: 13 - DNA, 63 bp, synthetic construct
  source 1..63: mol_type = other DNA
  modified_base 31: mod_base = OTHER; note = n is amino modified base
cctcgcatgactcaactgcctggtgatacgnggatgggcatgctcttcccgacggtattggac

SEQ ID NO: 14 - DNA, 63 bp, synthetic construct
  source 1..63: mol_type = other DNA
  modified_base 33: mod_base = OTHER; note = n is amino modified base
cttgctacacgattcagacttaggaatgttcgncatgcgagggtccaataccgacgattacag

SEQ ID NO: 15 - DNA, 63 bp, synthetic construct
  source 1..63: mol_type = other DNA
  modified_base 31: mod_base = OTHER; note = n is amino modified base
ggtgataaaacgtgtagcaagctgtaatcgncgggaagagcatgcccatccactactatggcg

SEQ ID NO: 16 - DNA, 30 bp, synthetic construct
  source 1..30: mol_type = other DNA
cagtctgattgcatcgttagctgtagatcg

SEQ ID NO: 17 - DNA, 20 bp, synthetic construct
  source 1..20: mol_type = other DNA
tagatcagtctgtaagcgcc

SEQ ID NO: 18 - DNA, 20 bp, synthetic construct
  source 1..20: mol_type = other DNA
ggcgcttacagactgatcta

SEQ ID NO: 19 - DNA, 40 bp, synthetic construct
  source 1..40: mol_type = other DNA
tagatcagtctgtaagcgccgttagcggtatagcccgtta

SEQ ID NO: 20 - DNA, 40 bp, synthetic construct
  source 1..40: mol_type = other DNA
taacgggctataccgctaacggcgcttacagactgatcta

SEQ ID NO: 21 - DNA, 60 bp, synthetic construct
  source 1..60: mol_type = other DNA
tagatcagtctgtaagcgccgttagcggtatagcccgttatcggctaggaaaacgcctag

SEQ ID NO: 22 - DNA, 60 bp, synthetic construct
  source 1..60: mol_type = other DNA
ctaggcgttttcctagccgataacgggctataccgctaacggcgcttacagactgatcta